SEQUENCE LISTING (157 sequences)

SEQ ID NO: 1 - protein, 12 aa, Artificial Sequence
  Synthetic Construct
KPLAVQKVFGSG

SEQ ID NO: 2 - protein, 10 aa, Artificial Sequence
  Synthetic Construct
RQAYLFGGSG

SEQ ID NO: 3 - protein, 13 aa, Artificial Sequence
  Synthetic Construct
YRLNLKHPVGGSG

SEQ ID NO: 4 - protein, 12 aa, Artificial Sequence
  Synthetic Construct
RLQYLFKSDGSG

SEQ ID NO: 5 - protein, 11 aa, Artificial Sequence
  Synthetic Construct
RYYPAFSGGSG

SEQ ID NO: 6 - protein, 12 aa, Artificial Sequence
  Synthetic Construct
RWQYYNQKFGSG

SEQ ID NO: 7 - protein, 12 aa, Artificial Sequence
  Synthetic Construct
KHSFQKELGGSG

SEQ ID NO: 8 - protein, 12 aa, Artificial Sequence
  Synthetic Construct
RYNLYKPSDGSG

SEQ ID NO: 9 - protein, 12 aa, Artificial Sequence
  Synthetic Construct
YRNAAWFSGGSG

SEQ ID NO: 10 - protein, 11 aa, Artificial Sequence
  Synthetic Construct
PRYQNFGGGSG

SEQ ID NO: 11 - protein, 14 aa, Artificial Sequence
  Synthetic Construct
RYPWNKLYKFDGSG

SEQ ID NO: 12 - protein, 14 aa, Artificial Sequence
  Synthetic Construct
YRFAQYKGQKDGSG

SEQ ID NO: 13 - protein, 9 aa, Artificial Sequence
  Synthetic Construct
RWYKNGGSG

SEQ ID NO: 14 - protein, 11 aa, Artificial Sequence
  Synthetic Construct
RWSFPAKGGSG

SEQ ID NO: 15 - protein, 13 aa, Artificial Sequence
  Synthetic Construct
YRPYGPVYNGGSG

SEQ ID NO: 16 - protein, 12 aa, Artificial Sequence
  Synthetic Construct
RFGQNLPLGGSG

SEQ ID NO: 17 - protein, 13 aa, Artificial Sequence
  Synthetic Construct
RAQYAQYLFGGSG

SEQ ID NO: 18 - protein, 15 aa, Artificial Sequence
  Synthetic Construct
RQYQKHSYAKFSGSG

SEQ ID NO: 19 - protein, 12 aa, Artificial Sequence
  Synthetic Construct
RYAYFQHLGGSG

SEQ ID NO: 20 - protein, 14 aa, Artificial Sequence
  Synthetic Construct
YRFQSGLPAKDGSG

SEQ ID NO: 21 - protein, 13 aa, Artificial Sequence
  Synthetic Construct
PRSFGYNNVGGSG

SEQ ID NO: 22 - protein, 12 aa, Artificial Sequence
  Synthetic Construct
RWRYYWKSEGSG

SEQ ID NO: 23 - protein, 7 aa, Artificial Sequence
  Synthetic Construct
RNWYGGS

SEQ ID NO: 24 - protein, 12 aa, Artificial Sequence
  Synthetic Construct
RWKYQKFNGGSG

SEQ ID NO: 25 - protein, 13 aa, Artificial Sequence
  Synthetic Construct
YRFPNAQSPGGSG

SEQ ID NO: 26 - protein, 11 aa, Artificial Sequence
  Synthetic Construct
RLYPQGYGGSG

SEQ ID NO: 27 - protein, 12 aa, Artificial Sequence
  Synthetic Construct
WRGFKFADGGSG

SEQ ID NO: 28 - protein, 11 aa, Artificial Sequence
  Synthetic Construct
RFKYPLDGGSG

SEQ ID NO: 29 - protein, 11 aa, Artificial Sequence
  Synthetic Construct
PRYSGQYGGSG

SEQ ID NO: 30 - protein, 12 aa, Artificial Sequence
  Synthetic Construct
YNRNAKSSGGSG

SEQ ID NO: 31 - protein, 12 aa, Artificial Sequence
  Synthetic Construct
KEAPGPRFGGSG

SEQ ID NO: 32 - protein, 13 aa, Artificial Sequence
  Synthetic Construct
EDEGLSKFEGGSG

SEQ ID NO: 33 - protein, 14 aa, Artificial Sequence
  Synthetic Construct
KNRPYNKDLEDGSG

SEQ ID NO: 34 - protein, 13 aa, Artificial Sequence
  Synthetic Construct
YSVDYWYQVDGSG

SEQ ID NO: 35 - protein, 16 aa, Artificial Sequence
  Synthetic Construct
VKDGANKHYWRVSGSG

SEQ ID NO: 36 - protein, 10 aa, Artificial Sequence
  Synthetic Construct
DLDKAFGGSG

SEQ ID NO: 37 - protein, 12 aa, Artificial Sequence
  Synthetic Construct
VERASYNLDGSG

SEQ ID NO: 38 - protein, 10 aa, Artificial Sequence
  Synthetic Construct
RPLQVQRGGS

SEQ ID NO: 39 - protein, 14 aa, Artificial Sequence
  Synthetic Construct
SVLEGYQAVFGGSG

SEQ ID NO: 40 - protein, 11 aa, Artificial Sequence
  Synthetic Construct
RSHPKHNVGSG

SEQ ID NO: 41 - protein, 11 aa, Artificial Sequence
  Synthetic Construct
QLLQLLFGGSG

SEQ ID NO: 42 - protein, 12 aa, Artificial Sequence
  Synthetic Construct
LAKQNRVEDGSG

SEQ ID NO: 43 - protein, 11 aa, Artificial Sequence
  Synthetic Construct
AFYGLELGGSG

SEQ ID NO: 44 - protein, 12 aa, Artificial Sequence
  Synthetic Construct
YVWGLELSGGSG

SEQ ID NO: 45 - protein, 9 aa, Artificial Sequence
  Synthetic Construct
LLGFLGGSG

SEQ ID NO: 46 - protein, 13 aa, Artificial Sequence
  Synthetic Construct
YSALFGLQEDGSG

SEQ ID NO: 47 - protein, 15 aa, Artificial Sequence
  Synthetic Construct
EGYANKLLALFSGSG

SEQ ID NO: 48 - protein, 12 aa, Artificial Sequence
  Synthetic Construct
EAVQKLLFSGSG

SEQ ID NO: 49 - protein, 13 aa, Artificial Sequence
  Synthetic Construct
LDRLGEYQFSGSG

SEQ ID NO: 50 - protein, 12 aa, Artificial Sequence
  Synthetic Construct
ELFGALFKGGSG

SEQ ID NO: 51 - protein, 13 aa, Artificial Sequence
  Synthetic Construct
VLSLNQVYSEGSG

SEQ ID NO: 52 - protein, 14 aa, Artificial Sequence
  Synthetic Construct
NVSDLSQFYLSGSG

SEQ ID NO: 53 - protein, 13 aa, Artificial Sequence
  Synthetic Construct
FGKLYQLYNDGSG

SEQ ID NO: 54 - protein, 11 aa, Artificial Sequence
  Synthetic Construct
LKQLFLEGGSG

SEQ ID NO: 55 - protein, 10 aa, Artificial Sequence
  Synthetic Construct
VVQALFDGSG

SEQ ID NO: 56 - protein, 12 aa, Artificial Sequence
  Synthetic Construct
FQFGKVVSDGSG

SEQ ID NO: 57 - protein, 14 aa, Artificial Sequence
  Synthetic Construct
VFGVLSQVARDGSG

SEQ ID NO: 58 - protein, 11 aa, Artificial Sequence
  Synthetic Construct
DLFQLVFSGSG

SEQ ID NO: 59 - protein, 15 aa, Artificial Sequence
  Synthetic Construct
WLDLGVFPYQHLGSG

SEQ ID NO: 60 - protein, 14 aa, Artificial Sequence
  Synthetic Construct
AQVAVDGFYVDGSG

SEQ ID NO: 61 - protein, 12 aa, Artificial Sequence
  Synthetic Construct
WRLNPLQFGGSG

SEQ ID NO: 62 - protein, 12 aa, Artificial Sequence
  Synthetic Construct
PRFNWKPYGGSG

SEQ ID NO: 63 - protein, 13 aa, Artificial Sequence
  Synthetic Construct
YWRFYANWAQLFG

SEQ ID NO: 64 - protein, 13 aa, Artificial Sequence
  Synthetic Construct
YQRHQNVFAKSGG

SEQ ID NO: 65 - protein, 12 aa, Artificial Sequence
  Synthetic Construct
WRPFWKNLGGSG

SEQ ID NO: 66 - protein, 12 aa, Artificial Sequence
  Synthetic Construct
ARWQQYKFGGSG

SEQ ID NO: 67 - protein, 12 aa, Artificial Sequence
  Synthetic Construct
YRYGWQKYGGSG

SEQ ID NO: 68 - protein, 12 aa, Artificial Sequence
  Synthetic Construct
YRGAGKNKGGSG

SEQ ID NO: 69 - protein, 12 aa, Artificial Sequence
  Synthetic Construct
RQRYPKVSGGSG

SEQ ID NO: 70 - protein, 13 aa, Artificial Sequence
  Synthetic Construct
RVLPYHGWKWFSG

SEQ ID NO: 71 - protein, 12 aa, Artificial Sequence
  Synthetic Construct
YRWGAKHYGGSG

SEQ ID NO: 72 - protein, 13 aa, Artificial Sequence
  Synthetic Construct
PQKSNHFHEAQKR

SEQ ID NO: 73 - protein, 12 aa, Artificial Sequence
  Synthetic Construct
YRRFPYFPGGSG

SEQ ID NO: 74 - protein, 12 aa, Artificial Sequence
  Synthetic Construct
YRRPWKFRDGSG

SEQ ID NO: 75 - protein, 13 aa, Artificial Sequence
  Synthetic Construct
WRVQAWRPNKVEG

SEQ ID NO: 76 - protein, 12 aa, Artificial Sequence
  Synthetic Construct
NRKFYWNAGGSG

SEQ ID NO: 77 - protein, 12 aa, Artificial Sequence
  Synthetic Construct
WRSVYVPNGGSG

SEQ ID NO: 78 - protein, 13 aa, Artificial Sequence
  Synthetic Construct
PVDFGYQLKVSGS

SEQ ID NO: 79 - protein, 13 aa, Artificial Sequence
  Synthetic Construct
WHLLGWVGYGGSG

SEQ ID NO: 80 - protein, 13 aa, Artificial Sequence
  Synthetic Construct
LFDNVLEFVEDGS

SEQ ID NO: 81 - protein, 13 aa, Artificial Sequence
  Synthetic Construct
PVPWRHVNYAHSG

SEQ ID NO: 82 - protein, 11 aa, Artificial Sequence
  Synthetic Construct
SGVLQFFGGSG

SEQ ID NO: 83 - protein, 13 aa, Artificial Sequence
  Synthetic Construct
KEDKVFGFRFDGS

SEQ ID NO: 84 - protein, 13 aa, Artificial Sequence
  Synthetic Construct
GYHLFEKLYFDGS

SEQ ID NO: 85 - protein, 13 aa, Artificial Sequence
  Synthetic Construct
NHLLEGAFVSEGS

SEQ ID NO: 86 - protein, 13 aa, Artificial Sequence
  Synthetic Construct
ALDKLSGLWKHVG

SEQ ID NO: 87 - protein, 9 aa, Artificial Sequence
  Synthetic Construct
FLQFLGGSG

SEQ ID NO: 88 - protein, 13 aa, Artificial Sequence
  Synthetic Construct
FYNFGYQDLEDGS

SEQ ID NO: 89 - protein, 13 aa, Artificial Sequence
  Synthetic Construct
EGFGYLFAHVGGS

SEQ ID NO: 90 - protein, 13 aa, Artificial Sequence
  Synthetic Construct
DYFEGQLNHLGGS

SEQ ID NO: 91 - protein, 13 aa, Artificial Sequence
  Synthetic Construct
FNKVLEYKWLFEG

SEQ ID NO: 92 - protein, 12 aa, Artificial Sequence
  Synthetic Construct
HVYGLNLFDGSG

SEQ ID NO: 93 - protein, 13 aa, Artificial Sequence
  Synthetic Construct
DANLFGYFKDGGS

SEQ ID NO: 94 - protein, 13 aa, Artificial Sequence
  Synthetic Construct
AFNVWQYFEGGSG

SEQ ID NO: 95 - protein, 12 aa, Artificial Sequence
  Synthetic Construct
VLAYKLQVDGSG

SEQ ID NO: 96 - protein, 13 aa, Artificial Sequence
  Synthetic Construct
VSWVFGLGHEGGS

SEQ ID NO: 97 - protein, 13 aa, Artificial Sequence
  Synthetic Construct
AWVELEYQYVFEG

SEQ ID NO: 98 - protein, 9 aa, Artificial Sequence
  Synthetic Construct
RWYKKWQPP

SEQ ID NO: 99 - protein, 9 aa, Artificial Sequence
  Synthetic Construct
RYFPYKPPQ

SEQ ID NO: 100 - protein, 9 aa, Artificial Sequence
  Synthetic Construct
RYYKNKPPP

SEQ ID NO: 101 - protein, 9 aa, Artificial Sequence
  Synthetic Construct
RYYKKGPPP

SEQ ID NO: 102 - protein, 9 aa, Artificial Sequence
  Synthetic Construct
RYFPFKPPP

SEQ ID NO: 103 - protein, 9 aa, Artificial Sequence
  Synthetic Construct
RYYYNKPPP

SEQ ID NO: 104 - protein, 9 aa, Artificial Sequence
  Synthetic Construct
RFFPYKPPQ

SEQ ID NO: 105 - protein, 9 aa, Artificial Sequence
  Synthetic Construct
RWWKKWQPP

SEQ ID NO: 106 - protein, 9 aa, Artificial Sequence
  Synthetic Construct
RYYYFKPNP

SEQ ID NO: 107 - protein, 9 aa, Artificial Sequence
  Synthetic Construct
RYYYYKPPP

SEQ ID NO: 108 - protein, 9 aa, Artificial Sequence
  Synthetic Construct
RFQPYKPPQ

SEQ ID NO: 109 - protein, 9 aa, Artificial Sequence
  Synthetic Construct
RYYKQKPPP

SEQ ID NO: 110 - protein, 9 aa, Artificial Sequence
  Synthetic Construct
RYFNYKPPQ

SEQ ID NO: 111 - protein, 9 aa, Artificial Sequence
  Synthetic Construct
RYYKYNPNP

SEQ ID NO: 112 - protein, 9 aa, Artificial Sequence
  Synthetic Construct
RYYPYKPPP

SEQ ID NO: 113 - protein, 9 aa, Artificial Sequence
  Synthetic Construct
RYYNYKPPP

SEQ ID NO: 114 - protein, 9 aa, Artificial Sequence
  Synthetic Construct
RWYKYGNPP

SEQ ID NO: 115 - protein, 9 aa, Artificial Sequence
  Synthetic Construct
RFYPYKPPP

SEQ ID NO: 116 - protein, 9 aa, Artificial Sequence
  Synthetic Construct
RYYFKNPNP

SEQ ID NO: 117 - protein, 9 aa, Artificial Sequence
  Synthetic Construct
RYYWYKPPP

SEQ ID NO: 118 - protein, 9 aa, Artificial Sequence
  Synthetic Construct
RYFPQKPGP

SEQ ID NO: 119 - protein, 9 aa, Artificial Sequence
  Synthetic Construct
RYYPFKPPP

SEQ ID NO: 120 - protein, 9 aa, Artificial Sequence
  Synthetic Construct
RWYYKKQGN

SEQ ID NO: 121 - protein, 9 aa, Artificial Sequence
  Synthetic Construct
RYYPQKPGP

SEQ ID NO: 122 - protein, 9 aa, Artificial Sequence
  Synthetic Construct
RYYYKNPNP

SEQ ID NO: 123 - protein, 9 aa, Artificial Sequence
  Synthetic Construct
RWFKKWQPP

SEQ ID NO: 124 - protein, 9 aa, Artificial Sequence
  Synthetic Construct
RYFKNKPPQ

SEQ ID NO: 125 - protein, 9 aa, Artificial Sequence
  Synthetic Construct
RYFNFKPPQ

SEQ ID NO: 126 - protein, 9 aa, Artificial Sequence
  Synthetic Construct
RYYNKNPGP

SEQ ID NO: 127 - protein, 9 aa, Artificial Sequence
  Synthetic Construct
RFYYNKPPP

SEQ ID NO: 128 - protein, 9 aa, Artificial Sequence
  Synthetic Construct
QLLQLLFGL

SEQ ID NO: 129 - protein, 9 aa, Artificial Sequence
  Synthetic Construct
FLLQLLFGG

SEQ ID NO: 130 - protein, 9 aa, Artificial Sequence
  Synthetic Construct
QVLQLLFGL

SEQ ID NO: 131 - protein, 9 aa, Artificial Sequence
  Synthetic Construct
GLLQLLFGL

SEQ ID NO: 132 - protein, 9 aa, Artificial Sequence
  Synthetic Construct
SVLEGYFAV

SEQ ID NO: 133 - protein, 9 aa, Artificial Sequence
  Synthetic Construct
VLLQLLFGP

SEQ ID NO: 134 - protein, 9 aa, Artificial Sequence
  Synthetic Construct
GVLQLLFGQ

SEQ ID NO: 135 - protein, 9 aa, Artificial Sequence
  Synthetic Construct
NLLQLLFGV

SEQ ID NO: 136 - protein, 9 aa, Artificial Sequence
  Synthetic Construct
NVLQLLFGV

SEQ ID NO: 137 - protein, 9 aa, Artificial Sequence
  Synthetic Construct
QLLQFLFLV

SEQ ID NO: 138 - protein, 9 aa, Artificial Sequence
  Synthetic Construct
FVLQLLFGL

SEQ ID NO: 139 - protein, 9 aa, Artificial Sequence
  Synthetic Construct
FLGQLLFGV

SEQ ID NO: 140 - protein, 9 aa, Artificial Sequence
  Synthetic Construct
SLLQLLFLP

SEQ ID NO: 141 - protein, 9 aa, Artificial Sequence
  Synthetic Construct
QVLQFLFLV

SEQ ID NO: 142 - protein, 9 aa, Artificial Sequence
  Synthetic Construct
QLLGLLFGG

SEQ ID NO: 143 - protein, 9 aa, Artificial Sequence
  Synthetic Construct
FLVQLLFGG

SEQ ID NO: 144 - protein, 9 aa, Artificial Sequence
  Synthetic Construct
ELLQLLFLV

SEQ ID NO: 145 - protein, 9 aa, Artificial Sequence
  Synthetic Construct
EVLQLLFLV

SEQ ID NO: 146 - protein, 9 aa, Artificial Sequence
  Synthetic Construct
VVLQLLFGQ

SEQ ID NO: 147 - protein, 9 aa, Artificial Sequence
  Synthetic Construct
QLLQSLFLW

SEQ ID NO: 148 - protein, 9 aa, Artificial Sequence
  Synthetic Construct
SVLEQYFAV

SEQ ID NO: 149 - protein, 9 aa, Artificial Sequence
  Synthetic Construct
PLLQLLFGL

SEQ ID NO: 150 - protein, 9 aa, Artificial Sequence
  Synthetic Construct
GLLGLLFGW

SEQ ID NO: 151 - protein, 9 aa, Artificial Sequence
  Synthetic Construct
QVLGLLFGL

SEQ ID NO: 152 - protein, 9 aa, Artificial Sequence
  Synthetic Construct
GVLGLLFGW

SEQ ID NO: 153 - protein, 9 aa, Artificial Sequence
  Synthetic Construct
LLLQLLFGQ

SEQ ID NO: 154 - protein, 9 aa, Artificial Sequence
  Synthetic Construct
SLLEGYFAV

SEQ ID NO: 155 - protein, 9 aa, Artificial Sequence
  Synthetic Construct
LVLQLLFGQ

SEQ ID NO: 156 - protein, 9 aa, Artificial Sequence
  Synthetic Construct
QLVQLLFGL

SEQ ID NO: 157 - protein, 9 aa, Artificial Sequence
  Synthetic Construct
DVLQLLFGV

(19) United States
(12) Patent Application Publication
(10) Pub. No.: US 2019/0064177 A1
(43) Pub. Date: Feb. 28, 2019

(54) INCORPORATING COMPETITIVE BINDING INFORMATION INTO MOLECULAR ARRAY ANALYSIS TO GENERATE FUNCTION NETWORKS OF INTERACTIONS

(71) Applicant: ARIZONA BOARD OF REGENTS ON BEHALF OF ARIZONA STATE UNIVERSITY, Scottsdale, AZ (US)

(72) Inventor: Neal Woodbury, Tempe, AZ (US)

(21) Appl. No.: 16/112,238

(22) Filed: Aug. 24, 2018

Related U.S. Application Data
(60) Provisional application No. 62/549,592, filed on Aug. 24, 2017.

(51) Int. Cl. G01N 33/68

(52) U.S. Cl. CPC G01N 33/6845

(57) ABSTRACT

Embodiments of a method are disclosed for using molecular arrays (peptide arrays) to define networks of functionally linked molecules. This is accomplished via competition experiments in which molecules from the array are added to solution, specifically inhibiting the function (e.g. binding of an antibody) of the similar molecule in the array as well as any other molecules in the array that would have the same function. These networks of functionally linked molecules are very useful in both understanding the chemical nature of the function and in improving the statistical robustness of functional detection on the array, e.g. defining a robust immunosignature in an immunosignature application.

### CROSS-REFERENCE TO RELATED APPLICATIONS

The present application claims priority to U.S. Provisional Patent Application No. 62/549,592, filed on Aug. 24, 2017, the disclosure of which is incorporated by reference in its entirety.

### BACKGROUND

Molecular arrays have proven to be a rich source of functional information about molecular systems. However, there are limitations associated with the analysis of arrays containing large numbers of heteropolymers that sparsely cover the total possible sequence space. Functional interactions between molecules or mixtures of molecules and molecular arrays often results in complex patterns of interactions and the relationship between the kinds of interactions of different molecules on the arrays with a sample is unclear. In order to cover sequence space as well as possible, these arrays typically contain more than 10⁵ different sequences. However, when looking for a statistically relevant binding event (e.g., that differs between disease and non-disease), this means that one must have statistical p-values of less than 10⁻⁵ to have any assurance that the effect is not just noise. There may well be very good differentiators with p-values of 10⁻³ that go undetected. Thus, a means of finding the peptides that share a common binding site and between them have much more statistically robust responses than any one alone is needed.

### SUMMARY

Embodiments herein relate to methods for detecting a common functional characteristic among a plurality of molecules through competitive binding. In one example, a method includes contacting a ligand with a target molecule, increasing the concentration of the ligand, and then detecting a level of binding between the target molecule and an array before and after the increasing step. If there is a decrease in a detection signal between the target molecule and one or more molecules on the array, it indicates a common functional characteristic between the ligand and the relevant array molecules and also a common functional characteristic among those array molecules. Accordingly, in some embodiments, methods are described for detecting a plurality of molecules (such as peptides) sharing a common characteristic in an array.

Further disclosed are embodiments relating to methods for detecting a plurality of molecules sharing a common functional characteristic among patient samples of different phenotypic states using competitive binding.

### BRIEF DESCRIPTION OF THE DRAWINGS

FIG. 1 depicts a scatter plot of diaphorase binding with or without Cofactor FAD.

FIG. 2 shows that adenine in FAD looks similar to tryptophan.

FIG. 3 depicts peptide sequences from an array and the corresponding ratios between 1 micromolar Diaphorase bound with and without FAD. The scatter plot compares the predicted and measured values.

### DETAILED DESCRIPTION

Embodiments of the disclosed technology describe an approach for using molecular array functional data based on molecular recognition in combination with selective competition using either known binding partners or array elements known to bind in order to sort the functional data into structurally distinct classes of interactions.

This technology disclosed herein is described in one or more exemplary embodiments in the following description. Reference throughout this specification to “one embodiment,” “an embodiment,” or similar language means that a particular feature, structure, or characteristic described in connection with the embodiment is included in at least one embodiment of the present technology disclosed herein. Thus, appearances of the phrases “in one embodiment,” “in an embodiment,” and similar language throughout this specification may, but do not necessarily, all refer to the same embodiment.

The described features, structures, or characteristics of the technology disclosed herein may be combined in any suitable manner in one or more embodiments. In the following description, numerous specific details are recited to provide a thorough understanding of embodiments of the technology disclosed herein. One skilled in the relevant art will recognize, however, that the technology disclosed herein may be practiced without one or more of the specific details, or with other methods, components, materials, and so forth. In other instances, well-known structures, materials, or operations are not shown or described in detail to avoid obscuring aspects of the technology disclosed herein.

The immunosignature technology involves observing the binding of total IgG (or another antibody isotype) to a sparse sampling of peptide sequences (or in principle, any heteropolymer sequences) in an array format. In many of the applications of this technology, what one does is compare many samples from individuals with one indication/phenotype to a set of individuals that have another indication (or are healthy). This concept can be extended beyond immune globins. Essentially any ligand or mixture of ligands can be bound to an array of peptides (heteropolymers) with the goal of determining the pattern of binding associated with the ligand(s) under a particular set of circumstances.

The immunosignature technology can be used to determine a pattern of binding that is indicative of the presence of a particular ligand, combination of ligands, disease state, health condition, physical condition, or environmental condition, etc. that results in a change in what molecules in a solution bind to the peptide (or other heteropolymer) array. Further, the immunosignature technology can be used to identify specific chemical features involved in the binding by using the information of what peptides (or heteropolymers) in the array are involved in the binding event in question. For example, one can determine what amino acid sequences an antibody binds to and in so doing generate information about what proteins/antigens/epitopes those antibodies are raised to bind to in blood. Or one might use this approach to find heteropolymer sequences involved in other protein-ligand interactions of biomedical or environmental importance.

In certain embodiments, the disclosed method is used to characterize monoclonal antibodies with peptide arrays. For example, incubating a monoclonal antibody that has a known linear epitope with a large peptide array yields many different binding events. Some of those binding events are with peptides that have the cognate sequence or sequences obviously similar to the cognate sequence. In other cases, it is often unclear what the relationship, if any, is with the cognate sequence. One can generate a network map in the following manner. First, if the epitope is known, this can be added to the solution. The cognate epitope will bind at the paratope on the antibody and if it is at sufficient concentration, it will block those sites from binding to peptides on the array. All of the peptides on the array that would otherwise bind to that site less tightly than the epitope will be effected and their signals should drop. This provides a specific map of the peptide sequences that occupy the same site as the cognate sequence. If the monoclonal antibody is itself a drug, this could indicated possible sequences of cross reactivity with proteins other than the intended target. A similar analysis can be performed even if the cognate epitope is not known. Individual peptides that show strong binding to the monoclonal antibody can be synthesized and added to the solution at increasing concentrations. Again this allows one to categorize the molecular recognition into groups that bind at the same site, creating a binding network, showing which peptides on the array are related in their site of interaction. This can be important in understanding noncognate interactions or analyzing antibodies that actually bind to the antigen at multiple, separated sites.

In certain embodiments, the disclosed method is used to characterize other proteins with peptide arrays. For example, proteins other than monoclonal antibodies can be characterized in terms of their molecular recognition or functional properties. Again, this can either be done by competition through solution binding with the known binding partner for the protein or by using the molecules on the array itself and mapping which array molecules compete effectively with which other molecules on the array, grouping them and creating a network of molecules with either similar of different molecular recognition properties relative to each other.

In certain embodiments, the disclosed method is used to characterize complex solutions such as blood. Consider the serum from a patient infected with a virus. Again, competition with the virus or viral components can allow one to find specific peptides (molecular array elements) that bind to the same site as the virus antigens that gave rise to an IgG or IgM response, for example. One can also compare serum samples of infected and uninfected individuals and use either known antigens or peptides that are identified as different between infected and uninfected as competitors. This will result in grouping of peptides that bind competitively to specific sites on the virus. This is an effective way to map epitopes on the viral proteome by performing the competition with different peptides, recording the sequences of the peptides that compete with that peptide, and using those sequences to identify peptide sequences in the viral proteome likely to be responsible for initiating the immune response.

This can easily be expanded to other diseases or conditions in which there are two sets of serum, with the disease or condition and without. Such conditions can also include response to therapy. In some embodiments, the peptide groups identified as binding to specifically are possible biomarkers. In other embodiments, they may be possible drug leads or targets for drug action. This is also an approach to identifying antigens, epitopes or molecular systems that could be components of vaccines or of immunotherapy drugs.

In certain embodiments, the disclosed method is used to enhance diagnosis. If one has two sets of serum from patients in one of two (or more) clinical relevant groups, the competition assay can be used to enhance the ability of molecular arrays to be used as a means of diagnosis, prognosis or treatment specification. One of the big issues in using large molecular arrays in diagnostics is the problem of feature selection. This is prevalent in approaches such as immunosignature analysis in which a large array of peptides selected to evenly cover peptide sequence space sparsely is bound to multiple samples from individuals in the categories that need to be distinguished. The goal of the immunosignature analysis is to accurately distinguish the patients in one class from those in another. The peptide arrays used in this method are large (10⁵ peptides to 10⁶ peptides, typically).

Generally one uses some type of statistical test on data from a group of samples that allows one to select a group of features to be used in classification. However, statistical methods of feature selection have two problems. First, they often find features in large arrays that are different between groups simply by statistical chance, but actually are not indicative of a true disease-based difference. Second, in large peptide arrays, many very significant differences can be buried in the noise. For example, in an array of 10⁵ elements, a p-value of 10⁻³ would be insignificant relative to statistical measures, and yet that might be a peptide that shows a very valuable and significant difference between the sample sets. By grouping peptides together and treating them as groups statistically, the ability to pull out features with true differences between classes is greatly improved, both in terms of avoiding features that arise do to noise and features that have good distinction but a lower statistical ranking in a very large dataset than would normally be required for feature selection.

Non-Limiting Working Example: Finding Peptides that Block or Favor Cofactor FAD Binding to Diaphorase.

Diaphorase is an NADP-depending dehydrogenase with a variety of substrate targets including some simple dye molecules. For this reason, it is often used as an indicator of NADPH (or NADH) concentration because it can reduce certain dyes changing their optical properties. One of its required cofactors is FAD. The bound FAD picks up electrons and protons from NAD(P)H and then transfers them to the product. Binding of FAD is reversible, so that often the enzyme as purchased either partially or entirely lacks FAD.

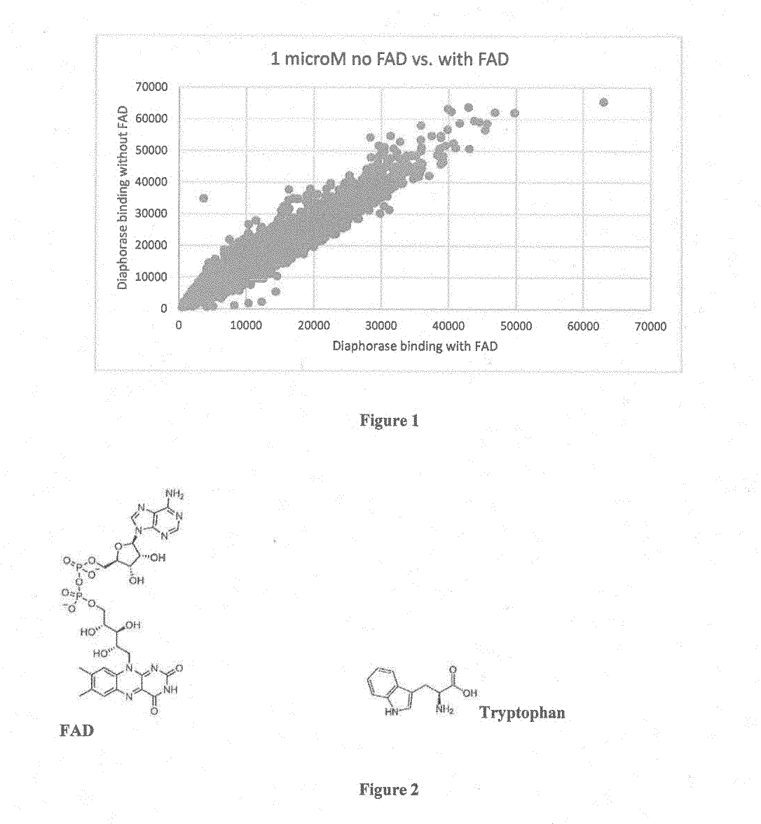

Measurements were performed in which diaphorase was bound to an array of about 126,000 unique sequences. The sequences were chosen simply to cover sequence space. 16 of the natural 20 amino acids were used to make up these sequences with C, I, M and T omitted. The diaphorase was fluorescently labeled and each peptide sequence feature was scanned and fluorescence recorded on the array. Diaphorase bound tightly to some peptides and not at all to others. Two different concentrations of diaphorase were used, 1 and 10 micromolar, and the diaphorase was either bound in the presence of excess FAD or not. Most of the binding values did not change upon addition of FAD. Indeed the Pearson correlation coefficient between w/ and w/o FAD was >0.97, which actually speaks to the reproducibility of this dataset. However, there are some differences as shown on the scatter plot of FIG. 1.

When one takes a ratio of the binding without FAD and the binding with FAD, one finds the following top 30 ratios (Table 1):

| | 1 microM | 1 micro | 10 microM | 10 microM | 1 microM | 10 microM |
|---|---|---|---|---|---|---|
| Sequence | FAD | no FAD | FAD | no FAD | noFAD/FAD | noFAD/FAD |
| KPLAVQKVGSG | 3707.5 | 34797.5 | 36814 | 46161 | 9.38570465 | 1.25389797 |
| (Seq. ID No. 1) | | | | | | |
| RQAYLFGGSG (Seq. | 4477 | 14479.75 | 64033.5 | 65535 | 3.23425285 | 1.02344866 |
| ID No. 2) | | | | | | |
| YRLNLKHPVGGSG | 4146.5 | 12652.75 | 56689 | 65535 | 3.05142892 | 1.15604438 |
| (Seq. ID No. 3) | | | | | | |
| RLQYLFKSDGSG | 1964.75 | 5778.25 | 30591 | 64328.25 | 2.94095941 | 2.10284888 |
| (Seq. ID No. 4) | | | | | | |
| RYYPAFSGGSG (Seq. | 7535.25 | 21898 | 65535 | 65535 | 2.90607478 | 1 |
| ID No. 5) | | | | | | |
| RWQYYNQKFGSG | 5458 | 15697.5 | 65535 | 65535 | 2.8760535 | 1 |
| (Seq. ID No. 6) | | | | | | |
| KHSFQKELGGSG | 727.5 | 2086 | 300.5 | 2387.25 | 2.86735395 | 0.79429379 |
| (Seq. ID No. 7) | | | | | | |
| RYNLYKPSDGSG | 1245.75 | 3569.5 | 18165 | 50327.75 | 2.86534216 | 2.77058904 |
| (Seq. ID No. 8) | | | | | | |
| YRNAAWFSGGSG | 2926 | 8372.25 | 43282 | 65535 | 2.86132946 | 1.51413983 |
| (Seq. ID No. 9) | | | | | | |
| PRYQNFGGGSG | 2458 | 6907 | 36662 | 65535 | 2.81000814 | 1.78754569 |
| (Seq. ID No. 10) | | | | | | |
| RYPWNKLYKFDGSG | 3483.25 | 9735.75 | 53014.5 | 65535 | 2.79501902 | 1.23617124 |
| (Seq. ID No. 11) | | | | | | |
| YRFAQYKGQKDGSG | 1604.25 | 4467.75 | 19933.75 | 56915 | 2.78494624 | 2.85520788 |
| (Seq. ID No. 12) | | | | | | |
| RWYKNGGSG (Seq. | 3658 | 10176.5 | 46265.5 | 64775 | 2.78198469 | 1.40007133 |
| ID No. 13) | | | | | | |
| RWSFPAKGGSG | 3614 | 10002.75 | 51221.25 | 65535 | 2.76777809 | 1.27944945 |
| (Seq. ID No. 14) | | | | | | |
| YRPYGPVYNGGSG | 6762.5 | 18709.5 | 65535 | 65535 | 2.76665434 | 1 |
| (Seq. ID No. 15) | | | | | | |
| RFGQNLPLGGSG | 2103.25 | 5802.25 | 34712 | 63502.5 | 2.75870676 | 1.82941058 |
| (Seq. ID No. 16) | | | | | | |
| RAQYAQYLFGGSG | 3162 | 8721.5 | 55433.5 | 65535 | 2.75822264 | 1.18222735 |
| (Seq. ID No. 17) | | | | | | |
| RQYQKHSYAKFSGSG | 2460 | 6757 | 27275.5 | 57203.5 | 2.74674797 | 2.09724845 |
| (Seq. ID No. 18) | | | | | | |
| RYAYFQHLGGSG | 4971.5 | 13584.75 | 64053.5 | 65535 | 2.73252539 | 1.0231291 |
| (Seq. ID No. 19) | | | | | | |
| YRFQSGLPAKDGSG | 1489.75 | 4041.25 | 20226.25 | 50316.25 | 2.71270347 | 2.48767072 |
| (Seq. ID No. 20) | | | | | | |
| PRSFGYNNVGGSG | 2525.5 | 6842.5 | 40495.5 | 65535 | 2.70936448 | 1.61832796 |
| (Seq. ID No. 21) | | | | | | |
| RWRYYWKSEGSG | 4093.5 | 11043 | 51412.25 | 65535 | 2.69769146 | 1.27469621 |
| (Seq. ID No. 22) | | | | | | |
| RNWYGGSG (Seq. ID | 2940.75 | 7830.5 | 48587 | 65535 | 2.6627561 | 1.34881758 |
| No. 23) | | | | | | |
| RWKYQKFNGGSG | 3860.75 | 10165 | 46173.5 | 61301.5 | 2.63290811 | 1.32763382 |
| (Seq. ID No. 24) | | | | | | |
| YRFPNAQSPGGSG | 2373 | 6240 | 37721.5 | 65535 | 2.62958281 | 1.73733812 |
| (Seq. ID No. 25) | | | | | | |
| RLYPQGYGGSG (Seq. | 4541.5 | 11912 | 61747 | 65535 | 2.62292194 | 1.06134711 |
| ID No. 26) | | | | | | |
| WRGFKFADGGSG | 2156.5 | 5654.5 | 31464.5 | 58885 | 2.6220728 | 1.8714742 |
| (Seq. ID No. 27) | | | | | | |
| RFKYPLDGGSG (Seq. | 2199.25 | 5764.75 | 30389 | 55899.25 | 2.62123451 | 1.83945671 |
| ID No. 28) | | | | | | |
| PRYSGQYGGSG (Seq. | 3234.25 | 8432.75 | 45157.75 | 65535 | 2.60732782 | 1.45124591 |
| ID No. 29) | | | | | | |
| YNRNAKSSGGSG | 1546 | 4014 | 18565.25 | 41333.25 | 2.59637775 | 2.22637724 |
| (Seq. ID No. 30) | | | | | | |

Note that all these sequences end with a GSG linker sequence. Because there are two concentrations, one can easily check for consistency. Note that 65535 is detector saturation, so one has to ignore those values. For this reason, the lower concentration is used most of the time. However, the very highest ratio, ˜9.4, does not show saturation in the high concentration array. It appears that this was some kind of error and was ignored in subsequent analysis. A similar conclusion can be made with the 7^{th} highest value. Looking at the sequences, RW or at least R or K followed by a nonpolar or aromatic amino acid at the N-terminus. This is interesting as FAD is negatively charged, though the adenine in FAD looks a bit like tryptophan (see FIG. 2).

There are also some peptides that appear to bind more strongly in the presence of FAD and thus would presumably stabilize the binding (Table 2):

| | 1 microM | 1 micro | 10 microM | 10 microM | 1 microM | 10 microM |
|---|---|---|---|---|---|---|
| Sequence | FAD | no FAD | FAD | no FAD | noFAD/FAD | noFAD/FAD |
| KEAPGPRFGGSG | 8281.5 | 1071.25 | 5467.5 | 5614.5 | 0.12935459 | 1.02688615 |
| (Seq. ID No. 31) | | | | | | |
| EDEGLSKFEGGSG | 4165.5 | 560.55 | 1378.5 | 1220.25 | 0.13455768 | 0.88520131 |
| (Seq. ID No. 32) | | | | | | |
| KNRPYNKDLEDGSG | 5147 | 733.25 | 2589.25 | 2522.5 | 0.14246163 | 0.97422033 |
| (Seq. ID No. 33) | | | | | | |
| YSVDYWYQVDGSG | 10415 | 1802.5 | 19218.25 | 19784.75 | 0.17306769 | 1.02947719 |
| (Seq. ID No. 34) | | | | | | |
| VKDGNKHYWRVSGSG | 12347.25 | 2221 | 17803.5 | 20355.75 | 0.17987811 | 1.14335664 |
| (Seq. ID No. 35) | | | | | | |
| DLDKAFGGSG | 2049.75 | 607.25 | 2142.25 | 1853 | 0.29625564 | 0.86497841 |
| (Seq. ID No. 36) | | | | | | |
| VERASYNLDGSG | 2238 | 759 | 3374 | 2900.5 | 0.33914209 | 0.85966212 |
| (Seq. ID No. 37) | | | | | | |
| RPLQVQRGGSG | 14421.5 | 5406 | 41059.5 | 53243 | 0.37485698 | 1.29672792 |
| (Seq. ID No. 38) | | | | | | |
| SVLEGYQAVFGGSG | 2664.5 | 1341.5 | 21517.25 | 6065.25 | 0.50347157 | 0.28187849 |
| (Seq. ID No. 39) | | | | | | |
| RSHPKHNVGSG | 3876 | 1953.667 | 12238.917 | 17018.417 | 0.50404197 | 1.39051659 |
| (Seq. ID No. 40) | | | | | | |
| QLLQLLFGGSG (Seq. ID | 6311.5 | 3228.25 | 53382.25 | 19529.5 | 0.51148697 | 0.36584258 |
| No. 41) | | | | | | |
| LAKQNRVEDGSS (Seq. | 1435 | 770.5 | 3699 | 2948.5 | 0.5369338 | 0.79710733 |
| ID No. 42 | | | | | | |
| AFYGLELGGSG (Seq. ID | 4182.5 | 2264.25 | 36729 | 16544 | 0.54136282 | 0.45043426 |
| No. 43) | | | | | | |
| YVWGLELSGGSG (Seq. | 4688.5 | 2607.5 | 36320 | 17286.5 | 0.55614802 | 0.47594989 |
| ID No. 44) | | | | | | |
| LLGFLGGSG (Seq. ID | 4143.5 | 2317.75 | 40264.25 | 26253 | 0.5593701 | 0.65201761 |
| No. 45) | | | | | | |
| YSALFGLQEDGSG (Seq. | 2100.75 | 1225.5 | 16872.25 | 8529 | 0.58336308 | 0.5055046 |
| ID No. 46) | | | | | | |
| EGYANKLLALFSGSG | 1727 | 1034 | 10408.25 | 6648 | 0.59872611 | 0.63872409 |
| (Seq. ID No. 47) | | | | | | |
| EAVQKLLFSGSG (Seq. | 2420 | 1457.5 | 17702.75 | 5389.5 | 0.60227273 | 0.30444422 |
| ID No. 48) | | | | | | |
| LDRLGEYQFSGSG (Seq. | 1164.75 | 709.25 | 7142.5 | 2995.75 | 0.60892895 | 0.41942597 |
| ID No. 49) | | | | | | |
| ELFGALFKGGSG (Seq. | 3403 | 2083.5 | 25315 | 12943.5 | 0.61225389 | 0.51129765 |
| ID No. 50) | | | | | | |
| VLSLNQVYSEGSG (Seq. | 2705.5 | 1674.75 | 21822.25 | 9853.75 | 0.61901682 | 0.45154601 |
| ID No. 51) | | | | | | |
| NVSDLSQFYLSGSG (Seq. | 3436.25 | 2144.5 | 27800.25 | 6739 | 0.62408148 | 0.24240789 |
| ID No. 52) | | | | | | |
| FGKLYQLYNDGSG (Seq. | 3049 | 1911.25 | 34155 | 15703 | 0.62684487 | 0.45975699 |
| ID No. 53) | | | | | | |
| LKQLFLEGGSG (Seq. ID | 2474.5 | 1559.25 | 23548.25 | 8696.5 | 0.6301273 | 0.36930557 |
| No. 54) | | | | | | |
| VVQALFDGSG (Seq. ID | 2304.25 | 1467.5 | 21180.25 | 9478.25 | 0.63686666 | 0.44750416 |
| No. 55) | | | | | | |
| FQFGKVVSDGSG (Seq. | 2470 | 1574.5 | 20187.75 | 12735.5 | 0.63744939 | 0.63085287 |
| ID No. 56) | | | | | | |
| VFGVLSQVARDGSG | 6638 | 4250.5 | 51495.5 | 26186.5 | 0.64032841 | 0.50852016 |
| (Seq. ID No. 57) | | | | | | |
| DLFQLVFSGSG (Seq. ID | 9011 | 5775 | 65535 | 32672.5 | 0.64088336 | 0.49855039 |
| No. 58) | | | | | | |
| WLDLGVFPYQHLGSG | 3433.25 | 2202 | 33610.5 | 25700.5 | 0.64137479 | 0.76465688 |
| (Seq. ID No. 59) | | | | | | |
| AQVAVDGFYVDGSG | 3089 | 2012.5 | 22739.5 | 12627 | 0.65150534 | 0.55528925 |
| (Seq. ID No. 60) | | | | | | |

A careful look at the top 10 of the top 12 again reveals large disagreement between the two concentrations tested. Thus, these are unlikely to actually bind more strongly in the presence of FAD. The remaining peptides are good candidates for stabilization of FAD binding.

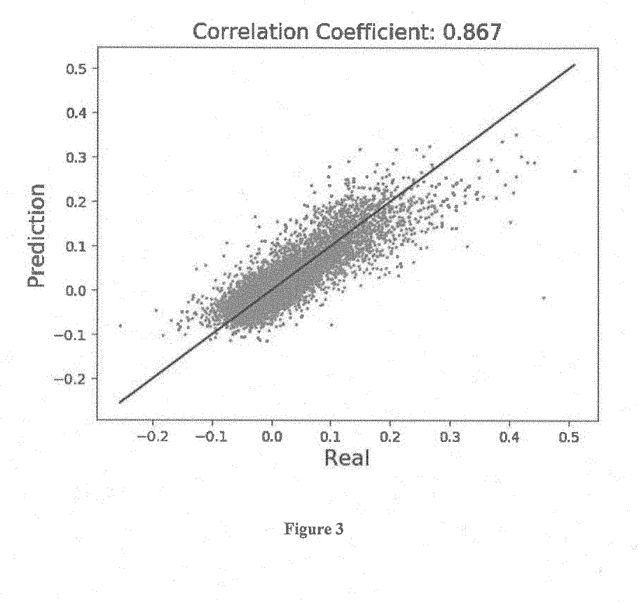

The ˜126,000 peptide sequences from the array and the corresponding ratios between 1 micromolar Diaphorase bound with and without FAD were fed into an algorithm that related the amino acid sequence of each sequence to its +/−FAD ratio of diaphorase binding. 90% of the sequences were used to train the model and 10% were left out as the test case. There is a reasonable relationship between the ratios and the sequences. This is difficult, because the datasets are really very similar so the noise on the ratios is accentuated. For the fit the log of the ratio was used because the noise is more or less log normal. The correlation coefficient comparing predictions of the 10% of the sequences NOT used in the training set with the measured ratios was 0.87. A scatter plot comparing the predicted and measured values is given in FIG. 3. Note that the 11 values in the tables above that were determined to be experimental artifact before fitting were removed (however those are the only values out of all ˜126,000 that were removed).

Thus, at least over this range, one can predict the relative binding with and without FAD reasonably well. The best measured values to their predicted values were compared in the table below (Table 3):

| | | Pred | Scamb | Scramb | Pred./Scram | Pred/Scram | Meas. |
|---|---|---|---|---|---|---|---|
| Sequence | pl | noFAD/FAD | mean ratio | ratio std | Zscore | ratio | ratio |
| KPLAVQKVFGSG | 10.81 | 1.35 | 1.27 | 1.09 | 0.07 | 1.06 | 9.39 |
| (Seq. ID No. 1) | | | | | | | |
| RQAYLFGGSG | 9.35 | 1.78 | 1.27 | 1.12 | 0.45 | 1.40 | 3.23 |
| (Seq. ID No. 2) | | | | | | | |
| YRLNLKHPVGGSG | 10.46 | 2.35 | 1.24 | 1.12 | 0.99 | 1.90 | 3.05 |
| (Seq. ID No. 3) | | | | | | | |
| RLQYLFKSDGSG | 9.30 | 1.82 | 1.17 | 1.13 | 0.58 | 1.56 | 2.94 |
| (Seq. ID No. 4) | | | | | | | |
| RYYPAFSGGSG | 9.17 | 2.33 | 1.34 | 1.16 | 0.85 | 1.74 | 2.91 |
| (Seq. ID No. 5) | | | | | | | |
| RWQYYNQKFGSG | 10.01 | 2.44 | 1.52 | 1.16 | 0.80 | 1.61 | 2.88 |
| (Seq. ID No. 6) | | | | | | | |
| KHSFQKELGGSG | 9.54 | 0.99 | 0.99 | 1.05 | 0.01 | 1.01 | 2.87 |
| (Seq. ID No. 7) | | | | | | | |
| RYNLYKPSDGSG | 9.15 | 1.94 | 1.14 | 1.14 | 0.70 | 1.70 | 2.87 |
| (Seq. ID No. 8) | | | | | | | |
| YRNAAWFSGGSG | 9.35 | 2.35 | 1.20 | 1.16 | 0.99 | 1.95 | 2.86 |
| (Seq. ID No. 9) | | | | | | | |
| PRYQNFGGGSG | 9.35 | 2.48 | 1.21 | 1.15 | 1.10 | 2.05 | 2.81 |
| (Seq. ID No. 10) | | | | | | | |
| RYPWNKLYKFDGS | 9.93 | 2.49 | 1.43 | 1.17 | 0.90 | 1.74 | 2.80 |
| (Seq. ID No. 11) | | | | | | | |
| YRFAQYKGQKDGS | 9.93 | 2.31 | 1.35 | 1.14 | 0.85 | 1.71 | 2.78 |
| (Seq. ID No. 12) | | | | | | | |
| RWYKNGGSG | 10.45 | 2.59 | 1.44 | 1.13 | 1.01 | 1.80 | 2.78 |
| (Seq. ID No. 13) | | | | | | | |
| RWSFPAKGGSG | 11.65 | 2.16 | 1.37 | 1.12 | 0.71 | 1.58 | 2.77 |
| (Seq. ID No. 14) | | | | | | | |
| YRPYGPVYNGGSG | 9.07 | 1.59 | 1.24 | 1.19 | 0.29 | 1.28 | 2.77 |
| (Seq. ID No. 15) | | | | | | | |
| RFGQNLPLGGSG | 10.55 | 2.15 | 1.16 | 1.16 | 0.85 | 1.85 | 2.76 |
| (Seq. ID No. 16) | | | | | | | |
| RAQYAQYLFGGSG | 9.17 | 2.21 | 1.27 | 1.15 | 0.82 | 1.74 | 2.76 |
| (Seq. ID No. 17) | | | | | | | |
| RQYQKHSYAKFSG | 10.45 | 2.41 | 1.47 | 1.12 | 0.84 | 1.63 | 2.75 |
| (Seq. ID No. 18) | | | | | | | |
| RYAYFQHLGGSG | 9.17 | 2.12 | 1.22 | 1.14 | 0.79 | 1.74 | 2.73 |
| (Seq. ID No. 19) | | | | | | | |
| YRFQSGLPAKDGS | 9.30 | 2.40 | 1.12 | 1.14 | 1.13 | 2.15 | 2.71 |
| (Seq. ID No. 20) | | | | | | | |
| PRSFGYNNVGGSG | 9.35 | 1.90 | 1.15 | 1.14 | 0.66 | 1.66 | 2.71 |
| (Seq. ID No. 21) | | | | | | | |
| RWRYYWKSEGSG | 10.01 | 1.95 | 1.36 | 1.12 | 0.53 | 1.43 | 2.70 |
| (Seq. ID No. 22) | | | | | | | |
| RNWYGGSG (Seq. | 9.35 | 1.61 | 1.29 | 1.18 | 0.27 | 1.25 | 2.66 |
| ID No. 23) | | | | | | | |
| RWKYQKFNGGSG | 10.90 | 2.12 | 1.59 | 1.12 | 0.47 | 1.33 | 2.63 |
| (Seq. ID No. 24) | | | | | | | |
| YRFPNAQSPGGSG | 9.35 | 2.03 | 1.14 | 1.18 | 0.75 | 1.78 | 2.63 |
| (Seq. ID No. 25) | | | | | | | |
| RLYPQGYGGSG | 9.17 | 2.09 | 1.27 | 1.17 | 0.70 | 1.64 | 2.62 |
| (Seq. ID No. 26) | | | | | | | |
| WRGFKFADGGSG | 9.70 | 2.36 | 1.14 | 1.13 | 1.08 | 2.08 | 2.62 |
| (Seq. ID No. 27) | | | | | | | |
| RFKYPLDGGSG | 9.30 | 2.20 | 1.21 | 1.16 | 0.86 | 1.82 | 2.62 |
| (Seq. ID No. 28) | | | | | | | |
| PRYSGQYGGSG | 9.17 | 2.30 | 1.22 | 1.15 | 0.94 | 1.89 | 2.61 |
| (Seq. ID No. 29) | | | | | | | |
| YNRNAKSSGGSG | 10.45 | 2.48 | 1.30 | 1.15 | 1.02 | 1.90 | 2.60 |
| (Seq. ID No. 30) | | | | | | | |

For this table and those that follow, the columns are as follows: Sequence: The peptide sequence being considered

Pred. noFAD/FAD: The predicted value of the ratio between the sample without FAD over the sample with FAD.
Scramb. Mean Ratio: This is the mean ratio computed for 100 scrambled versions of the peptide sequence (same amino acid composition but different orders)
Scramb. Ratio std: This is the standard deviation of the ratio predicted for the 100 scrambled versions of the sequence
Pred./Scram. Zscore: This is the Z score representing how different the predicted value of the ratio is compared to the scrambled mean value (it is the difference in values divided by the standard deviation for the scrambled values
Pred./Scram. Ratio: This is the ratio between the predicted and scrambled mean values Meas. Ratio: This is the ratio between without and with FAD calculated from the measured binding values of the two samples.

In the table above, one can see a few things. First, the predicted value for the first and the seventh are way low, consistent with the conclusions from looking at different concentrations. Note that they are also effectively indistinguishable from a scrambled sequence mean.

If one instead looks for the peptides with the highest predicted scores (Table 4):

| | | Pred | Scamb | Scramb | Pred./Scram | Pred/Scram | Meas. |
|---|---|---|---|---|---|---|---|
| Sequence | pl | noFAD/FAD | mean ratio | ratio std | Zscore | ratio | ratio |
| WRLNPLQFGGSG | 10.55 | 2.61 | 1.15 | 1.13 | 1.29 | 2.28 | 2.54 |
| (Seq. ID No. 61) | | | | | | | |
| RWYKNGGSG (Seq. | 10.45 | 2.59 | 1.44 | 1.13 | 1.01 | 1.80 | 2.78 |
| ID No. 13) | | | | | | | |
| PRFNWKPYGGSG | 10.45 | 2.56 | 1.42 | 1.19 | 0.96 | 1.80 | 2.44 |
| (Seq. ID No. 62) | | | | | | | |
| YWRFYANWAQLFG | 9.17 | 2.50 | 1.29 | 1.16 | 1.04 | 1.94 | 2.56 |
| (Seq. ID No. 63) | | | | | | | |
| RYPWNKLYKFDGS | 9.93 | 2.49 | 1.43 | 1.17 | 0.90 | 1.74 | 2.80 |
| (Seq. ID No. 11) | | | | | | | |
| PRYQNFGGGSG | 9.35 | 2.48 | 1.21 | 1.15 | 1.10 | 2.05 | 2.81 |
| (Seq. ID No. 10) | | | | | | | |
| YNRNAKSSGGSG | 10.45 | 2.48 | 1.30 | 1.15 | 1.02 | 1.90 | 2.60 |
| (Seq. ID No. 30) | | | | | | | |
| YQRHQNVFAKSGG | 10.46 | 2.48 | 1.28 | 1.13 | 1.06 | 1.93 | 2.56 |
| (Seq. ID No. 64) | | | | | | | |
| RWQYYNQKFGSG | 10.01 | 2.44 | 1.52 | 1.16 | 0.80 | 1.61 | 2.88 |
| (Seq. ID No. 6) | | | | | | | |
| WRPFWKNLGGSG | 11.65 | 2.44 | 1.37 | 1.15 | 0.93 | 1.78 | 2.18 |
| (Seq. ID No. 65) | | | | | | | |
| ARWQQYKFGGSG | 10.45 | 2.42 | 1.41 | 1.12 | 0.90 | 1.72 | 2.18 |
| (Seq. ID No. 66) | | | | | | | |
| YRYGWQKYGGSG | 9.78 | 2.41 | 1.52 | 1.12 | 0.80 | 1.59 | 2.43 |
| (Seq. ID No. 67) | | | | | | | |
| RQYQKHSYAKFSG | 10.45 | 2.41 | 1.47 | 1.12 | 0.84 | 1.63 | 2.75 |
| (Seq. ID No. 18) | | | | | | | |
| YRFQSGLPAKDGS | 9.30 | 2.40 | 1.12 | 1.14 | 1.13 | 2.15 | 2.71 |
| (Seq. ID No. 20) | | | | | | | |
| YRGAGKNKGGSG | 10.90 | 2.40 | 1.46 | 1.12 | 0.83 | 1.64 | 1.49 |
| (Seq. ID No. 68) | | | | | | | |
| RQRYPKVSGGSG | 11.53 | 2.38 | 1.45 | 1.11 | 0.84 | 1.64 | 1.71 |
| (Seq. ID No. 69) | | | | | | | |
| RVLPYHGWKWFSG | 10.46 | 2.38 | 1.34 | 1.14 | 0.91 | 1.78 | 2.45 |
| (Seq. ID No. 70) | | | | | | | |
| WRGFKFADGGSG | 9.70 | 2.36 | 1.14 | 1.13 | 1.08 | 2.08 | 2.62 |
| (Seq. ID No. 27) | | | | | | | |
| YRLNLKHPVGGSG | 10.46 | 2.35 | 1.24 | 1.12 | 0.99 | 1.90 | 3.05 |
| (Seq. ID No. 3) | | | | | | | |
| YRNAAWFSGGSG | 9.35 | 2.35 | 1.20 | 1.16 | 0.99 | 1.95 | 2.86 |
| (Seq. ID No. 9) | | | | | | | |
| YRWGAKHYGGSG | 10.01 | 2.33 | 1.33 | 1.12 | 0.90 | 1.75 | 2.06 |
| (Seq. ID No. 71) | | | | | | | |
| PQKSNHFHEAQKR | 10.79 | 2.33 | 1.05 | 1.06 | 1.20 | 2.22 | 2.41 |
| (Seq. ID No. 72) | | | | | | | |
| RYYPAFSGGSG | 9.17 | 2.33 | 1.34 | 1.16 | 0.85 | 1.74 | 2.91 |
| (Seq. ID No. 5) | | | | | | | |
| YRFAQYKGQKDGS | 9.93 | 2.31 | 1.35 | 1.14 | 0.85 | 1.71 | 2.78 |
| (Seq. ID No. 12) | | | | | | | |
| YRRFPYFPGGSG | 10.12 | 2.31 | 1.50 | 1.14 | 0.71 | 1.54 | 1.60 |
| (Seq. ID No. 73) | | | | | | | |
| YRRPWKFRDGSG | 11.43 | 2.30 | 1.52 | 1.11 | 0.71 | 1.52 | 1.74 |
| (Seq. ID No. 74) | | | | | | | |
| PRYSGQYGGSG | 9.17 | 2.30 | 1.22 | 1.15 | 0.94 | 1.89 | 2.61 |
| (Seq. ID No. 29) | | | | | | | |
| WRVQAWRPNKVEG | 11.48 | 2.30 | 1.20 | 1.13 | 0.98 | 1.92 | 2.43 |
| (Seq. ID No. 75) | | | | | | | |
| NRKFYWNAGGSG | 10.45 | 2.29 | 1.44 | 1.15 | 0.74 | 1.59 | 2.10 |
| (Seq. ID No. 76) | | | | | | | |
| WRSVYVPNGGSG | 9.35 | 2.27 | 1.15 | 1.16 | 0.96 | 1.97 | 2.35 |
| (Seq. ID No. 77) | | | | | | | |

The results are similar, but actually there is more consistency between predicted and measured among the top peptides sorted this way. As before, R at the N-terminus plays a big role, K is also present in the middle or near the C-terminus of most sequences. There are also patterns with F, Y, L, W. Note that GSG on the C-terminus is a linker sequence common to all of the peptides, so that can be ignored in the comparison. The above peptides are reasonable candidates for ligands that would either displace FAD complete or at least would favor the unbound form of the enzyme.

Given below are the lowest measured ratios (peptides that potentially stabilize FAD binding) (Table 5):

| | | Pred | Scamb | Scramb | Pred./Scram | Pred/Scram | Meas. |
|---|---|---|---|---|---|---|---|
| Sequence | pl | noFAD/FAD | mean ratio | ratio std | Zscore | ratio | ratio |
| KEAPGPRFGGSG | 9.70 | 1.09 | 1.04 | 1.08 | 0.05 | 1.05 | 0.13 |
| (Seq. ID No. 31) | | | | | | | |
| EDEGLSKFEGCSG | 3.78 | 1.00 | 0.97 | 1.02 | 0.02 | 1.02 | 0.13 |
| (Seq. ID No. 32) | | | | | | | |
| KNRPYNKDLEOGS | 6.55 | 1.04 | 0.96 | 1.03 | 0.08 | 1.09 | 0.14 |
| (Seq. ID No. 33) | | | | | | | |
| YSVDYWYQVDGSG | 3.49 | 0.94 | 0.91 | 1.04 | 0.03 | 1.03 | 0.17 |
| (Seq. ID No. 34) | | | | | | | |
| VKDGANKHYWRVS | 10.24 | 1.12 | 1.15 | 1.03 | -0.03 | 0.97 | 0.18 |
| (Seq. ID No. 35) | | | | | | | |
| DLDKAFGGSG | 4.11 | 0.98 | 0.98 | 1.03 | 0.00 | 1.00 | 0.30 |
| (Seq. ID No. 36) | | | | | | | |
| VERASYNLQGSG | 4.18 | 0.98 | 0.96 | 1.05 | 0.02 | 1.02 | 0.34 |
| (Seq. ID No. 37) | | | | | | | |
| RPLQVQRGGSG | 12.50 | 1.44 | 1.35 | 1.10 | 0.08 | 1.07 | 0.37 |
| (Seq. ID No. 38) | | | | | | | |
| SVLEGYQAVFGGS | 3.85 | 0.42 | 0.92 | 1.07 | -0.46 | 0.46 | 0.50 |
| (Seq. ID No. 39) | | | | | | | |
| RSHPKHNVGSG | 11.65 | 1.21 | 1.12 | 1.07 | 0.08 | 1.08 | 0.50 |
| (Seq. ID No. 40) | | | | | | | |
| QLLQLLFGGSG | 6.10 | 0.54 | 0.95 | 1.09 | -0.37 | 0.57 | 0.51 |
| (Seq. ID No. 41) | | | | | | | |
| LAKQNRVEDGSG | 6.49 | 0.99 | 0.98 | 1.06 | 0.01 | 1.02 | 0.54 |
| (Seq. ID No. 42) | | | | | | | |
| AFYGLELGGSG | 3.85 | 0.74 | 0.93 | 1.06 | -0.18 | 0.80 | 0.54 |
| (Seq. ID No. 43) | | | | | | | |
| YVWGLELSGGSG | 3.85 | 0.90 | 0.92 | 1.06 | -0.02 | 0.98 | 0.56 |
| (Seq. ID No. 44) | | | | | | | |
| LLGFLGGSG (Seq. | 6.10 | 0.73 | 0.98 | 1.09 | -0.23 | 0.74 | 0.56 |
| ID No. 45) | | | | | | | |
| YSALFGLQEDGSG | 3.55 | 0.78 | 0.94 | 1.05 | -0.15 | 0.83 | 0.58 |
| (Seq. ID No. 46) | | | | | | | |
| EGYANKLLALFSG | 6.41 | 0.69 | 0.94 | 1.07 | -0.24 | 0.73 | 0.60 |
| (Seq. ID No. 47) | | | | | | | |
| EAVQKLLFSGSG | 6.41 | 0.83 | 0.95 | 1.06 | -0.12 | 0.87 | 0.60 |
| (Seq. ID No. 48) | | | | | | | |
| LDRLGEYQFSGSG | 4.18 | 0.65 | 0.94 | 1.07 | -0.27 | 0.69 | 0.61 |
| (Seq. ID No. 49) | | | | | | | |
| ELFGALFKGGSG | 6.41 | 0.79 | 0.95 | 1.06 | -0.16 | 0.83 | 0.61 |
| (Seq. ID No. 50) | | | | | | | |
| VLSLNQVYSEGSG | 3.85 | 0.78 | 0.92 | 1.05 | -0.14 | 0.84 | 0.62 |
| (Seq. ID No. 51) | | | | | | | |
| NVSDLSQFYLSGS | 3.75 | 0.68 | 0.91 | 1.06 | -0.22 | 0.74 | 0.62 |
| (Seq. ID No. 52) | | | | | | | |
| FGKLYQLYNDGSG | 6.32 | 0.80 | 0.96 | 1.07 | -0.15 | 0.84 | 0.63 |
| (Seq. ID No. 53) | | | | | | | |
| LKQLFLEGGSG | 6.41 | 0.84 | 0.94 | 1.06 | -0.09 | 0.90 | 0.63 |
| (Seq. ID No. 54) | | | | | | | |
| VVQALFDGSG | 3.75 | 0.97 | 0.93 | 1.05 | 0.04 | 1.04 | 0.64 |
| (Seq. ID No. 55) | | | | | | | |
| FQFGKVVSDGSG | 6.34 | 0.90 | 0.96 | 1.06 | -0.06 | 0.94 | 0.64 |
| (Seq. ID No. 56) | | | | | | | |
| VFGVLSQVARDGS | 6.34 | 0.74 | 1.00 | 1.12 | -0.23 | 0.74 | 0.64 |
| (Seq. ID No. 57) | | | | | | | |
| DLFQLVFSGSG | 3.75 | 0.78 | 0.90 | 1.07 | -0.11 | 0.87 | 0.64 |
| (Seq. ID No. 58) | | | | | | | |
| WLDLGVFPYQHLG | 5.29 | 0.71 | 0.88 | 1.08 | -0.16 | 0.80 | 0.64 |
| (Seq. ID No. 59) | | | | | | | |
| AQVAVDGFYVDGS | 3.49 | 0.69 | 0.93 | 1.05 | -0.22 | 0.75 | 0.65 |
| (Seq. ID No. 60) | | | | | | | |

As before, but this time based on predictions, one would guess the measured values for 10 of the first 12 are probably artifacts. The remainder are more consistent with a couple of exceptions. If one sorts to give the best predicted values (rather than measured) you get this (Table 6):

| | | Pred | Scamb | Scramb | Pred./Scram | Pred/Scram | Meas. |
|---|---|---|---|---|---|---|---|
| Sequence | pl | noFAD/FAD | mean ratio | ratio std | Zscore | ratio | ratio |
| SVLEGYQAVFGGS (Seq. | 3.85 | 0.42 | 0.92 | 1.07 | -0.46 | 0.46 | 0.50 |
| ID No. 39) | | | | | | | |
| QLLQLLFGGSG (Seq. ID | 6.10 | 0.54 | 0.95 | 1.09 | -0.37 | 0.57 | 0.51 |
| No. 41) | | | | | | | |
| LDRLGEYQFSGSG (Seq. | 4.18 | 0.65 | 0.94 | 1.07 | -0.27 | 0.69 | 0.61 |
| ID No. 49) | | | | | | | |
| NVSDLSQFYLSGS (Seq. | 3.75 | 0.68 | 0.91 | 1.06 | -0.22 | 0.74 | 0.62 |
| ID No. 52) | | | | | | | |
| EGYANKLLALFSG (Seq. | 6.41 | 0.69 | 0.94 | 1.07 | -0.24 | 0.73 | 0.60 |
| ID No. 47) | | | | | | | |
| AQVAVDGFYVDGS | 3.49 | 0.69 | 0.93 | 1.05 | -0.22 | 0.75 | 0.65 |
| (Seq. ID No. 60) | | | | | | | |
| WLDLGVFPYQHLG (Seq. | 5.29 | 0.71 | 0.88 | 1.08 | -0.16 | 0.80 | 0.64 |
| ID No. 59) | | | | | | | |
| PVDFGYQLKVSGS (Seq. | 6.33 | 0.71 | 0.94 | 1.07 | -0.21 | 0.76 | 0.66 |
| ID No. 78) | | | | | | | |
| LLGFLGGSG (Seq. ID | 6.10 | 0.73 | 0.98 | 1.09 | -0.23 | 0.74 | 0.56 |
| No. 45) | | | | | | | |
| AFYGLELGGSG (Seq. ID | 3.85 | 0.74 | 0.93 | 1.06 | -0.18 | 0.80 | 0.54 |
| No. 43) | | | | | | | |
| VFGVLSQVARDGS | 6.34 | 0.74 | 1.00 | 1.12 | -0.23 | 0.74 | 0.64 |
| (Seq. ID No. 57) | | | | | | | |
| WHLLGWVGYGGSG | 7.54 | 0.74 | 0.90 | 1.07 | -0.15 | 0.83 | 0.70 |
| (Seq. ID No. 79) | | | | | | | |
| LFDNVLEFVEDGS (Seq. | 3.29 | 0.75 | 0.93 | 1.04 | -0.18 | 0.80 | 0.69 |
| ID No. 80) | | | | | | | |
| PVPWRHVNYAHSG | 9.35 | 0.75 | 1.03 | 1.10 | -0.26 | 0.72 | 0.69 |
| (Seq. ID No. 81) | | | | | | | |
| SGVLQFFGGSG (Seq. ID | 6.10 | 0.75 | 0.98 | 1.09 | -0.22 | 0.76 | 0.70 |
| No. 82) | | | | | | | |
| KEDKVFGFRFDGS (Seq. | 6.56 | 0.75 | 0.96 | 1.06 | -0.20 | 0.78 | 0.70 |
| ID No. 83) | | | | | | | |
| GYHLFEKLYFDGS (Seq. | 5.45 | 0.75 | 0.90 | 1.06 | -0.14 | 0.83 | 0.70 |
| ID No. 84) | | | | | | | |
| NHLLEGAFVSEGS (Seq. | 4.25 | 0.75 | 0.94 | 1.04 | -0.18 | 0.80 | 0.77 |
| ID No. 85) | | | | | | | |
| ALDKLSGLWKHVG | 9.54 | 0.75 | 0.98 | 1.08 | -0.21 | 0.77 | 0.69 |
| (Seq. ID No. 86) | | | | | | | |
| FLQFLGGSG (Seq. ID | 6.10 | 0.75 | 1.00 | 1.09 | -0.23 | 0.75 | 0.87 |
| No. 87) | | | | | | | |
| FYNFGYQDLEDGS (Seq. | 3.38 | 0.76 | 0.92 | 1.04 | -0.16 | 0.82 | 0.75 |
| ID No. 88) | | | | | | | |
| EGFGYLFAHVGGS (Seq. | 5.36 | 0.76 | 0.90 | 1.07 | -0.14 | 0.84 | 0.78 |
| ID No. 89) | | | | | | | |
| DYFEGQLNHLGGS | 4.18 | 0.76 | 0.94 | 1.04 | -0.17 | 0.81 | 0.75 |
| (Seq. ID No. 90) | | | | | | | |
| FNKVLEYKWLFEG (Seq. | 6.52 | 0.76 | 0.91 | 1.07 | -0.14 | 0.83 | 0.73 |
| ID No. 91) | | | | | | | |
| HVYGLNLFDGSG (Seq. | 5.29 | 0.76 | 0.91 | 1.06 | -0.14 | 0.83 | 0.72 |
| ID No. 92) | | | | | | | |
| DANLFGYFKDGGS | 4.11 | 0.76 | 0.94 | 1.04 | -0.17 | 0.81 | 0.70 |
| (Seq. ID No. 93) | | | | | | | |
| AFNVWQYFEGGSG | 3.85 | 0.76 | 0.91 | 1.05 | -0.15 | 0.83 | 0.73 |
| (Seq. ID No. 94) | | | | | | | |
| VLAYKLQVDGSG (Seq. | 6.33 | 0.76 | 0.96 | 1.07 | -0.18 | 0.80 | 0.68 |
| ID No. 95) | | | | | | | |
| VSWVFGLGHEGGS | 5.36 | 0.76 | 0.92 | 1.04 | -0.15 | 0.83 | 0.71 |
| (Seq. ID No. 96) | | | | | | | |
| AWVELEYQYVFEG | 3.47 | 0.77 | 0.92 | 1.05 | -0.14 | 0.84 | 0.74 |
| (Seq. ID No. 97) | | | | | | | |

Again, sorting by the prediction, the high values are more consistent between predicted and measured. These peptides are good candidates for peptides that would favor the FAD bound form of the enzyme, presumably stabilizing it.

Because an algorithm has been developed to predict sequences that bind either favoring or disfavoring FAD binding, one can project this against any sequence, searching for sequences that potentially favor or disfavor binding more strongly. For the 16 amino acids used in the array, there are total of about 69 billion sequences possible. The ratio for each was calculated and 1 million of the best values and sequence were saved. These were not merely the top million. The problem with taking the true top million is that they tend to be very similar to each other. Instead the analysis was performed in an ordered fashion (AAAAAAAAA, AAAAAAAAD, AAAAAAAAE, AAAAAAAAF . . . ), working through the sequences in small groups and picking the best ratios from each group. In this way one is assured not to pick all top values from one local region of sequence space. The highest ratios are given below (peptides the bind only in the absence of FAD and thus presumably destabilize its binding), along with the values predicted for an average of many other sequences with the same amino acid composition (in principle, one wants sequence specific binding rather than binding based entirely on composition of the peptide) (Table 7):

| | | Pred. | Scamb. | Scram. | Pred/Scram | Pred/Scram |
|---|---|---|---|---|---|---|
| Sequence | pl | ratio | Mean ratio | std Ratio | Zscore | ratio |
| RWYKKWQPP | 10.90 | 3.96 | 1.64 | 0.17 | 13.67 | 2.41 |
| (Seq. ID No. | | | | | | |
| 98) | | | | | | |
| RYFPYKPPQ | 10.01 | 3.75 | 1.62 | 0.21 | 10.37 | 2.31 |
| (Seq. ID No. | | | | | | |
| 99) | | | | | | |
| RYYKNKPPP | 10.45 | 3.74 | 1.69 | 0.20 | 10.20 | 2.21 |
| (Seq. ID No. | | | | | | |
| 100) | | | | | | |
| RYYKKGPPP | 10.45 | 3.65 | 1.65 | 0.20 | 9.89 | 2.21 |
| (Seq. ID No. | | | | | | |
| 101) | | | | | | |
| RYFPFKPPP | 10.45 | 3.62 | 1.63 | 0.25 | 7.86 | 2.22 |
| (Seq. ID No. | | | | | | |
| 102) | | | | | | |
| RYYYNKPPP | 9.78 | 3.62 | 1.70 | 0.27 | 6.99 | 2.13 |
| (Seq. ID No. | | | | | | |
| 103) | | | | | | |
| RFFPYKPPQ | 10.45 | 3.62 | 1.60 | 0.20 | 9.99 | 2.27 |
| (Seq. ID No. | | | | | | |
| 104) | | | | | | |
| RWWKKWQPP | 11.82 | 3.60 | 1.63 | 0.18 | 11.22 | 2.21 |
| (Seq. ID No. | | | | | | |
| 105) | | | | | | |
| RYYYFKPNP | 9.78 | 3.59 | 1.70 | 0.23 | 8.13 | 2.11 |
| (Seq. ID No. | | | | | | |
| 106) | | | | | | |
| RYYYYKPPP | 9.63 | 3.58 | 1.71 | 0.21 | 8.72 | 2.09 |
| (Seq. ID No. | | | | | | |
| 107) | | | | | | |
| RFQPYKPPQ | 10.45 | 3.57 | 1.57 | 0.23 | 8.53 | 2.28 |
| (Seq. ID No. | | | | | | |
| 108) | | | | | | |
| RYYKQKPPP | 10.45 | 3.57 | 1.67 | 0.19 | 9.80 | 2.13 |
| (Seq. ID No. | | | | | | |
| 109) | | | | | | |
| RYFNYKPPQ | 10.01 | 3.56 | 1.62 | 0.20 | 9.55 | 2.20 |
| (Seq. ID No. | | | | | | |
| 110) | | | | | | |
| RYYKYNPNP | 9.78 | 3.55 | 1.73 | 0.30 | 6.19 | 2.06 |
| (Seq. ID No. | | | | | | |
| 111) | | | | | | |
| RYYPYKPPP | 9.78 | 3.54 | 1.71 | 0.28 | 6.63 | 2.07 |
| (Seq. ID No. | | | | | | |
| 112) | | | | | | |
| RYYNYKPPP | 9.78 | 3.54 | 1.72 | 0.28 | 6.59 | 2.06 |
| (Seq. ID No. | | | | | | |
| 113) | | | | | | |
| RWYKYGNPP | 10.01 | 3.54 | 1.61 | 0.24 | 7.99 | 2.19 |
| (Seq. ID No. | | | | | | |
| 114) | | | | | | |
| RFYPYKPPP | 10.01 | 3.53 | 1.68 | 0.26 | 7.14 | 2.11 |
| (Seq. ID No. | | | | | | |
| 115) | | | | | | |
| RYYFKNPNP | 10.01 | 3.53 | 1.67 | 0.28 | 6.62 | 2.12 |
| (Seq. ID No. | | | | | | |
| 116) | | | | | | |
| RYYWYKPPP | 9.78 | 3.53 | 1.69 | 0.23 | 8.02 | 2.09 |
| (Seq. ID No. | | | | | | |
| 117) | | | | | | |
| RYFPQKPGP | 10.45 | 3.53 | 1.56 | 0.23 | .60 | 2.26 |
| (Seq. ID No. | | | | | | |
| 118) | | | | | | |
| RYYPFKPPP | 10.01 | 3.52 | 1.68 | 0.27 | 6.77 | 2.10 |
| (Seq. ID No. | | | | | | |
| 119) | | | | | | |
| RWYYKKQGN | 10.45 | 3.51 | 1.60 | 0.18 | 10.67 | 2.19 |
| (Seq. ID No. | | | | | | |
| 120) | | | | | | |
| RYYPQKPGP | 10.01 | 3.51 | 1.60 | 0.26 | 7.41 | 2.19 |
| (Seq. ID No. | | | | | | |
| 121) | | | | | | |
| RYYYKNPNP | 9.78 | 3.51 | 1.73 | 0.29 | 6.09 | 2.03 |
| (Seq. ID No. | | | | | | |
| 122) | | | | | | |
| RWFKKWQPP | 11.82 | 3.51 | 1.61 | 0.16 | 12.23 | 2.18 |
| (Seq. ID No. | | | | | | |
| 123) | | | | | | |
| RYFKNKPPQ | 10.90 | 3.51 | 1.61 | 0.16 | 11.92 | 2.18 |
| (Seq. ID No. | | | | | | |
| 124) | | | | | | |
| RYFNFKPPQ | 10.45 | 3.50 | 1.58 | 0.20 | 9 .40 | 2.21 |
| (Seq. ID No. | | | | | | |
| 125) | | | | | | |
| RYYNKNPGP | 10.01 | 3.50 | 1.60 | 0.26 | 7.35 | 2.19 |
| (Seq. ID No. | | | | | | |
| 126) | | | | | | |
| RFYYNKPPP | 10.01 | 3.50 | 1.65 | 0.26 | 7.18 | 2.11 |
| (Seq. ID No. | | | | | | |
| 127) | | | | | | |

For the most part, this resembles what was seen from the best values on the array, though the prediction is that these values would be much higher for the ratio. The form seems to be R-aromatic stretch-NK-PQ. If one looks farther down, one finds that the KN and the PQ regions can be replaced with other sequences or that the K can be moved closer to the C-terminus. The R can also be in the second position at times (as seen for the array peptides). These are good candidates for peptides that destabilize FAD binding to diaphorase.

The peptides that stabilize FAD binding look like this (Table 8):

| | | Pred. | Scamb. | Scram. | Pred/Scram | Pred/Scram |
|---|---|---|---|---|---|---|
| Sequence | pl | ratio | Mean ratio | std Ratio | Zscore | ratio |
| QLLQLLFGL | 6.10 | 0.40 | 0.99 | 0.10 | -5.82 | 0.41 |
| (Seq. ID No. | | | | | | |
| 128) | | | | | | |
| FLLQLLFGG | 6.10 | 0.42 | 1.00 | 0.11 | -5.41 | 0.41 |
| (Seq. ID No. | | | | | | |
| 129) | | | | | | |
| QVLQLLFGL | 6.10 | 0.42 | 1.00 | 0.10 | -5.85 | 0.43 |
| (Seq. ID No. | | | | | | |
| 130) | | | | | | |
| GLLQLLFGL | 6.10 | 0.42 | 0.99 | 0.10 | -5.54 | 0.43 |
| (Seq. ID No. | | | | | | |
| 131) | | | | | | |
| SVLEGYFAV | 3.85 | 0.43 | 0.95 | 0.07 | -7.52 | 0.45 |
| (Seq. ID No. | | | | | | |
| 132) | | | | | | |
| VLLQLLFGP | 6.10 | 0.44 | 0.98 | 0.09 | -5.99 | 0.45 |
| (Seq. ID No. | | | | | | |
| 133) | | | | | | |
| GVLQLLFGQ | 6.10 | 0.44 | 1.00 | 0.10 | -5.81 | 0.44 |
| (Seq. ID No. | | | | | | |
| 134) | | | | | | |
| NLLQLLFGV | 6.10 | 0.44 | 0.98 | 0.09 | -6.10 | 0.45 |
| (Seq. ID No. | | | | | | |
| 135) | | | | | | |
| NVLQLLFGV | 6.10 | 0.44 | 0.99 | 0.09 | -6.41 | 0.45 |
| (Seq. ID No. | | | | | | |
| 136) | | | | | | |
| QLLQFLFLV | 6.10 | 0.44 | 1.03 | 0.11 | -5.49 | 0.43 |
| (Seq. ID No. | | | | | | |
| 137) | | | | | | |
| FVLQLLFGL | 6.10 | 0.45 | 1.01 | 0.10 | -5.41 | 0.45 |
| (Seq. ID No. | | | | | | |
| 138) | | | | | | |
| FLGQLLFGV | 6.10 | 0.45 | 1.00 | 0.10 | -5.39 | 0.45 |
| (Seq. ID No. | | | | | | |
| 139) | | | | | | |
| SLLQLLFLP | 6.10 | 0.46 | 1.01 | 0.10 | -5.36 | 0.45 |
| (Seq. ID No. | | | | | | |
| 140) | | | | | | |
| QVLQFLFLV | 6.10 | 0.46 | 1.05 | 0.10 | -6.06 | 0.44 |
| (Seq. ID No. | | | | | | |
| 141) | | | | | | |
| QLLGLLFGG | 6.10 | 0.46 | 0.99 | 0.09 | -5.82 | 0.46 |
| (Seq. ID No. | | | | | | |
| 142) | | | | | | |
| FLVQLLFGG | 6.10 | 0.46 | 1.01 | 0.10 | -5.40 | 0.46 |
| (Seq. ID No. | | | | | | |
| 143) | | | | | | |
| ELLQLLFLV | 3.85 | 0.46 | 0.93 | 0.07 | -6.61 | 0.50 |
| (Seq. ID No. | | | | | | |
| 144 | | | | | | |
| EVLQLLFLV | 3.85 | 0.46 | 0.94 | 0.08 | -6.37 | 0.49 |
| (Seq. ID No. | | | | | | |
| 145) | | | | | | |
| VVLQLLFGQ | 6.10 | 0.46 | 0.99 | 0.09 | -5.89 | 0.47 |
| (Seq. ID No. | | | | | | |
| 146) | | | | | | |
| QLLQSLFLW | 6.10 | 0.46 | 1.01 | 0.11 | -5.13 | 0.46 |
| (Seq. ID No. | | | | | | |
| 147) | | | | | | |
| SVLEQYFAV | 3.85 | 0.46 | 0.96 | 0.07 | -6.99 | 0.49 |
| (Seq. ID No. | | | | | | |
| 148) | | | | | | |
| PLLQLLFGL | 6.10 | 0.47 | 0.98 | 0.11 | -4.79 | 0.47 |
| (Seq. ID No. | | | | | | |
| 149) | | | | | | |
| GLLGLLFGW | 6.10 | 0.47 | 0.96 | 0.09 | -5.31 | 0.48 |
| (Seq. ID No. | | | | | | |
| 150) | | | | | | |
| QVLGLLFGL | 6.10 | 0.47 | 0.99 | 0.09 | -5.68 | 0.47 |
| (Seq. ID No. | | | | | | |
| 151) | | | | | | |
| GVLGLLFGW | 6.10 | 0.47 | 0.97 | 0.09 | -5.76 | 0.48 |
| (Seq. ID No. | | | | | | |
| 152) | | | | | | |
| LLLQLLFGQ | 6.10 | 0.47 | 0.99 | 0.10 | -5.16 | 0.47 |
| (Seq. ID No. | | | | | | |
| 153 | | | | | | |
| SLLEGYFAV | 3.85 | 0.47 | 0.95 | 0.07 | -6.94 | 0.50 |
| (Seq. ID No. | | | | | | |
| 154) | | | | | | |
| LVLQLLFGQ | 6.10 | 0.47 | 0.99 | 0.09 | -5.54 | 0.47 |
| (Seq. ID No. | | | | | | |
| 155) | | | | | | |
| QLVQLLFGL | 6.10 | 0.47 | 1.00 | 0.10 | -5.23 | 0.48 |
| (Seq. ID No. | | | | | | |
| 156) | | | | | | |
| DVLQLLFGV | 3.75 | 0.47 | 0.93 | 0.07 | -6.53 | 0.51 |
| (Seq. ID No. | | | | | | |
| 157) | | | | | | |

Two of the top peptides on the array for stabilizing FAD are essentially the same as two near the top here (see red/lighter sequences and compare to top two predicted values on the array above), so again, these peptides are suggesting similar rules as was seen with the array peptides. These are thus good candidates for optimized sequences that stabilize FAD binding to diaphorase.

While the preferred embodiments of the present technology have been illustrated in detail, it should be apparent that modifications and adaptations to those embodiments may occur to one skilled in the art without departing from the scope of the present technology.

What is claimed is:

1. A method for detecting a common functional characteristic between a ligand and one or more molecules on an array through competitive binding, comprising:
contacting a target molecule to an array of molecules;
increasing a concentration of a ligand to the target molecule, and
detecting a level of binding between said target molecule and said array of molecules before and after said increasing step, wherein,
a decrease in a detection signal between said target molecule and one or more molecules on said array indicates a common functional characteristic between said ligand and said one or more molecules on the array, while an increase in a detection signal between said target molecule and one or more molecules on said array indicates that the molecular recognition of said ligand is stabilized by said one or more molecules on the array.

2. The method of claim 1, wherein the ligand has a strong binding affinity to the target molecule's binding site.

3. The method of claim 2, wherein the ligand is cognate to the target molecule's binding site.

4. The method of claim 1, wherein said ligand is a peptide and said target molecule is a protein.

5. The method of claim 1, wherein said ligand is a viral antigen and said target molecule is a serum protein.

6. The method of claim 1, wherein said target molecule includes a detectable label.

7. A method for detecting a common functional characteristic among two or more molecules on an array through competitive binding, comprising:
contacting a target molecule to an array of molecules;
increasing a concentration of a ligand to the target molecule, and
detecting a level of binding between said target molecule and said array of molecules before and after said increasing step, wherein,
a decrease in a detection signal between said target molecule and two or more molecules on said array indicates a common functional characteristic among said two or more molecules on the array.

8. The method of claim 7, wherein the ligand has a strong binding affinity to the target molecule's binding site.

9. The method of claim 8, wherein the ligand is cognate to the target molecule's binding site.

10. The method of claim 7, wherein said ligand is a peptide and said target molecule is a protein.

11. The method of claim 7, wherein said ligand is a viral antigen and said target molecule is a serum protein.

12. The method of claim 7, wherein said target molecule includes a detectable label.

13. A method for detecting a plurality of peptides sharing a common molecular recognition function to an antibody, comprising:
contacting an antibody to a plurality of peptides on a peptide array,
increasing a concentration of an antigen known to bind to a paratope of said antibody; and
detecting a level of binding between said antibody and the peptides on the array before and after said increasing step; wherein,
a decrease in a detection signal between said antibody and two or more peptides on said array indicates a common molecular recognition function among said two or more peptides on the array.

14. The method of claim 13, wherein the antigen has a strong binding affinity to the antibody's paratope.

15. The method of claim 14, wherein the antigen is the antibody's cognate epitope.

16. The method of claim 13, wherein said antibody includes a detectable label.

17. A method for detecting a plurality of molecules sharing a common functional characteristic between patient samples of different phenotypic states using competitive binding, comprising:
contacting each of said patient samples to an array of molecules;
increasing a concentration of a ligand, and
detecting a level of binding between said patient samples and said array of molecules before and after said increasing step, wherein,
a decrease detection of a differential binding signal between said patient samples and two or more molecules on said array indicates a common functional characteristic among said two or more molecules on the array.

18. The method of claim 17, wherein said different phenotypic states comprise a healthy control and a disease phenotype.

19. The method of claim 18, wherein said different phenotypic states comprise two different disease phenotypes.

20. The method of claims 10, wherein said molecules sharing a common functional characteristic are grouped and classified to establish a diagnostic indication for said different phenotypic states.
